(19) United States
(12) Patent Application Publication
(10) Pub. No.: US 2018/0338993 A1
(43) Pub. Date: Nov. 29, 2018

(54) TREATMENT OF HEPATITIS DELTA VIRUS INFECTION

(71) Applicant: Eiger BioPharmaceuticals, Inc., Palo Alto, CA (US)

(72) Inventors: David Cory, Palo Alto, CA (US); Ingrid Choong, Palo Alto, CA (US); Jeffrey S. Glenn, Palo Alto, CA (US)

(73) Assignee: Eiger BioPharmaceuticals, Inc., Palo Alto, CA (US)

(21) Appl. No.: 15/531,695

(22) PCT Filed: Dec. 3, 2015
(86) PCT No.: PCT/US15/63674

§ 371 Date: May 30, 2017

Related U.S. Application Data
(60) Provisional application No. 62/087,692, filed on Dec. 4, 2014.

(51) Int. Cl. A61K 31/714; A61K 31/4196

(52) U.S. Cl. CPC A61K 31/714; A61K 31/4196

(57) ABSTRACT

Tipifarnib or a tipifarnib derivative is used to treat HDV infection as a monotherapy or in combination with an interferon and/or boosting agent such as a CYP3A4 inhibitor such as ritonavir and cobicistal. This invention arises in part out of the surprising discoveries that not all prenyltransferase inhibitors are efficacious in treating HDV infection and that tipifarnib (Rl 15777) and tipifarnib derivatives such as R208176 can be administered at a dose efficacious in humans. This invention accordingly provides a method of inhibiting HDV replication in a human subject known to be co-infected with HBV and HDV by administering a therapeutically effective dose of tipifarnib, R208176 and other therapeutically effective tipifarnib derivatives and pharmaceutically acceptable salts and other forms.

This application claims the benefit of U.S. Patent Application No. 62/087,692, filed Dec. 4, 2014, which is hereby incorporated by reference in its entirety for all purposes.

### FIELD

The present invention provides methods for treating viral hepatitis resulting from Hepatitis delta virus (HDV) infection, and so relates to the fields of chemistry, medicinal chemistry, medicine, molecular biology, and pharmacology.

### BACKGROUND

HDV causes the most severe form of viral hepatitis, and there is no effective medical therapy (see Lau, 1999, Hepatology 30:546-549). The HDV large delta antigen protein contains a CXXX box rendering it a substrate for prenylation (see Zhang and Casey, 1996, Annu. Rev. Biochem. 65:241-269) by the prenyl lipid farnesyl (see Glenn et al., 1992, Science 256:1331-1333, and Otto and Casey, 1996, J. Biol. Chem. 271:4569-4572). Farnesylation of proteins catalysed by FTase is an essential step in processing of a variety of proteins and occurs by transfer of the farnesyl group of farnesyl pyrophosphate to a cysteine at the C-terminal tetrapeptide of a protein in a structural motif sometimes referred to as the CAAX box. Further post-translational modifications of a farnesylated protein, including proteolytic cleavage at the cysteine residue of the CAAX box and methylation of the cysteine carboxyl, generally follow farnesylation. Molecular genetic experiments demonstrated that specific mutation of the prenylation site in large delta antigen prevents both its prenylation and HDV particle formation (see Glenn et al., 1992, supra). PCT Pub. No. WO 2011/088126, incorporated herein by reference, describes the potential of using prenyltransferase inhibitors in humans to treat HDV infection and suggests a number of different doses, dosing frequencies, and combination therapeutics to achieve efficacy. There continues to be an ongoing need for agents to treat HDV infection. U.S. provisional application Ser. No. 62/073,413, filed 31 Oct. 2014, incorporated herein by reference, describes the use of the prenyltransferase inhibitor lonafarnib in humans to treat HDV infection. There remains a need, however, for additional therapeutics to treat this deadly infection, both as single agent therapy and in combination with other agents, such as lonafarnib, interferon, and ritonavir. The present invention meets this need.

### SUMMARY

This invention arises in part out of the surprising discoveries that not all prenyltransferase inhibitors are efficacious in treating HDV infection and that tipifarnib (R115777) and tipifarnib derivatives such as R208176 can be administered at a dose efficacious in humans. This invention accordingly provides a method of inhibiting HDV replication in a human subject known to be co-infected with HBV and HDV by administering a therapeutically effective dose of tipifarnib, R208176 and other therapeutically effective tipifarnib derivatives and pharmaceutically acceptable salts and other forms.

In one embodiment, the method comprises orally administering a therapeutically effective dose of tipifarnib to a human subject known to be co-infected with HBV and HDV at a total daily dose of between 200 mg to 600 mg for a period of at least 30 consecutive days, or for at least about 60 or 90 days or longer. In various embodiments, tipifarnib is administered BID; for example and without limitation, tipifarnib can be administered at a dose of 100 mg BID for a total daily dose of 200 mg; 150 mg BID for a total daily dose of 300 mg; 200 mg BID for a total daily dose of 400 mg; 250 mg BID for a total daily dose of 500 mg; and 300 mg BID for a total daily dose of 600 mg. In various embodiments, tipifarnib is co-administered with lonafarnib and/or one or more drugs currently approved or otherwise used for treatment of HBV or HDV. In various embodiments, tipifarnib therapy is followed by or preceeded with lonafarnib or a currently approved or otherwise used therapy.

In another embodiment, the present invention provides pharmaceutical formulations and unit dose forms of the compounds and pharmaceutical formulations useful in the methods of the invention.

In another embodiment, the invention relates to the administration of tipifarnib and tipifarnib derivatives such as R208176 in combination with other agents, such as interferon alpha and ritonavir (Norvir. With such agents, efficacious therapy may be achieved at tipifarnib doses of 100 mg once (QD) or twice (BID) daily, 150 mg once (QD) or twice (BID) daily, 200 mg once (QD) or twice (BID) daily, 250 mg once (QD) or twice (BID) daily or 300 mg once (QD) or twice (BID) daily.

Thus, in one aspect, this invention provides a method of treating an HDV infection in a human, said method comprising administering to the human in need of such treatment: a daily dose of about 100 to 300 mg QD or BID of tipifarnib or a tipifarnib derivative such as R208176, or a pharmaceutically acceptable salt thereof, or a pharmaceutically acceptable solvate thereof, and a therapeutically effective amount of interferon-α, for at least about 30 days, or for at least about 60 or 90 days, thereby treating the HDV infection. In another aspect, this invention provides method of reducing an HDV-RNA viral load in a human, said method comprising administering to the human in need of such reduction a daily dose of about 100 mg to 300 mg QD or BID of tipifarnib or a tipifarnib derivative such as R208176, a pharmaceutically acceptable salt thereof, or a pharmaceutically acceptable solvate of each thereof, for at least about 60 days or at least about 90 days, wherein the viral load of HDV-RNA is reduced by at least 1, at least, 1.5, or by at least 2 log HDV-RNA copies/mL, thereby reducing the HDV-RNA viral load.

In another aspect, this invention provides method of reducing an HDV-RNA viral load in a human, said method comprising administering to the human in need of such reduction: a daily dose of about 100 mg to 300 mg QD or BID of tipifarnib or a tipifarnib derivative such as R208176, a pharmaceutically acceptable salt thereof, or a pharmaceutically acceptable solvate of each thereof, and a therapeutically effective amount of interferon-α, for at least about 30 days, or at least about 60 or at least about 90 days, wherein the load of HDV-RNA is reduced by at least 1, at least, 1.5, or by at least 2 log HDV-RNA copies/mL, thereby reducing the HDV-RNA viral load.

In some embodiments, tipifarnib (or a tipifarnib derivative such as R208176) treatment is continued for a period of time until HDV-RNA levels are below 3 log HDV-RNA copies/mL or 1,000 copies/mL. In some embodiments, the treatments are continued for at least about 30 days, at least about 60 days, at least about 90 days, at least about 120 days, or for the rest of a HDV patient's life. In some embodiments, tipifarnib (or a tipifarnib derivative such as R208176) is administered at a daily dose of 100 mg BID. In some embodiments, tipifarnib is administered at a daily dose of 150 mg BID. In some embodiments, tipifarnib (or a tipifarnib derivative such as R208176) is administered at a daily dose of 200 mg BID. In some embodiments, tipifarnib (or a tipifarnib derivative such as R208176) is administered at a daily dose of 250 mg BID. In some embodiments, tipifarnib (or a tipifarnib derivative such as R208176) is administered at a daily dose of 300 mg BID.

In some embodiments, both tipifarnib and interferon-α are administered to the patient; in some embodiments the tipifarnib dose is 100 mg to 300 mg QD or BID. In some embodiments, the administration of tipifarnib and the interferon-α is concurrent. In some embodiments, the administration of tipifarnib and the interferon-α is sequential. In some embodiments, the interferon-α is Pegasys. In some embodiments, the Pegasys is administered weekly. In some embodiments, the Pegasys is administered at a dose of 180 micrograms per week. In these embodiments, dosing of tipifarnib and the interferon is continued for at least 30 days, usually at least about 60 or even 90 days or longer, including 6 months to a year or longer. In some embodiments, dosing will be discontinued after virus levels have decreased to undetectable levels for a period of time (such as 1 to 3 months or longer).

In some embodiments, both tipifarnib (or a tipifarnib derivative such as R208176) and ritonavir or similar boosting agent are administered to the patient; in some embodiments the tipifarnib dose is 100 mg to 300 mg QD or BID, and the ritonavir dose is 100 mg QD; in some embodiments the tipifarnib dose is 100 to 300 mg QD or BID and the ritonavir dose is 50 mg BID. In these embodiments, dosing of tipifarnib and the ritonavir or other boosting agent is continued for at least 30 days, usually at least about 60 or even 90 days or longer, including 6 months to a year or longer. In some embodiments, dosing will be discontinued after virus levels have decreased to undetectable levels for a period of time (such as 1 to 3 months or longer).

These and other aspects and embodiments of the invention are described in more detail below.

### DETAILED DESCRIPTION

This detailed description of the invention is divided into sections for the convenience of the reader. In Section I, definitions of terms used herein are provided. In Section II, treatment of HDV infection in accordance with the methods of the invention is described. In Section III, pharmaceutical compositions and unit dose forms useful in accordance with the methods of the invention are described. In Section IV, methods for administering tipifarnib (and tipifarnib derivatives such as R208176), pharmaceutical compositions, and unit dose forms useful in accordance with the methods of the invention are described. In Section V, combination therapies of the invention are described. In Section VI, combination therapies for ameliorating GI distress that may occur with tipifarnib (or a tipifarnib derivative such as R208176) dosing are described. This section is followed by examples illustrating the anti-viral activity of tipifarnib when used in accordance with the invention.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible. Embodiments of the present disclosure will employ, unless otherwise indicated, techniques of synthetic organic chemistry, biochemistry, biology, molecular biology, recombinant DNA techniques, pharmacology, and the like, which are within the skill of the art. Such techniques are explained fully in the literature. This disclosure is not limited to particular embodiments described, and the embodiment of the invention in practice may, of course, vary from that described herein.

### I. Definitions

The terminology used herein is for the purpose of describing particular embodiments only, and is not intended to be limiting, because the scope of the present invention will be limited only by the appended claims. Unless defined otherwise, all technical and scientific terms used herein have the same meaning as commonly understood by one of ordinary skill in the art to which this invention belongs. In this specification and in the claims that follow, reference will be made to a number of terms that shall be defined to have the following meanings unless a contrary intention is apparent. In some cases, terms with commonly understood meanings are defined herein for clarity and/or for ready reference, and the inclusion of such definitions herein should not be construed as representing a substantial difference over the definition of the term as generally understood in the art.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All technical and patent publications cited herein are incorporated herein by reference in their entirety. Nothing herein is to be construed as an admission that the invention is not entitled to antedate such disclosure by virtue of prior invention.

All numerical designations, e.g., pH, temperature, time, concentration, and molecular weight, including ranges, are approximations which are varied (+) or (−) by increments of 0.1 or 1.0, as appropriate. It is to be understood, although not always explicitly stated that all numerical designations are preceded by the term “about”.

The singular forms “a,” “an,” and “the” include plural referents unless the context clearly dictates otherwise. Thus, for example, reference to “a compound” includes a plurality of compounds.

The term “administration” refers to introducing a compound, a composition, or an agent of the present disclosure into a host, such as a human. One preferred route of administration of the agents is oral administration. Another preferred route is intravenous administration. However, any route of administration, such as topical, subcutaneous, peritoneal, intraarterial, inhalation, vaginal, rectal, nasal, introduction into the cerebrospinal fluid, or instillation into body compartments can be used.

The term “comprising” is intended to mean that the compounds, compositions and methods include the recited elements, but not excluding others. “Consisting essentially of” when used to define compounds, compositions and methods, shall mean excluding other elements that would materially affect the basic and novel characteristics of the claimed invention. “Consisting of” shall mean excluding any element, step, or ingredient not specified in the claim. Embodiments defined by each of these transition terms are within the scope of this invention.

The term “tipifarnib”, also known under the trade name Zarnestra® (J&JPRD), refers to an FTase inhibitor (R)-6-[amino(4-chlorophenyl)(1-methyl-1H-imidazol-5-yl)methyl]-4-(3-chlorophenyl)-1-methyl-2(1H)-quinolinone (also identified as R115777) having the structure shown below:

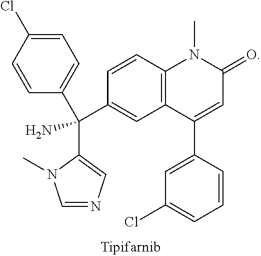 [SMILES: Cn1cncc1[C@@](N)(c1ccc(Cl)cc1)c1ccc2c(c1)c(-c1cccc(Cl)c1)cc(=O)n2C]

Tipifarnib is a methyl-quinolinone with a molecular formula of C₂₇H₂₂Cl₂N₄O and molecular weight of 489.40. Tipifarnib is insoluble in water, citrate-NaOH buffer, and phosphate buffers; slightly soluble in citrate-HCl buffer, and moderately soluble in 0.1 N HCl. In addition to the drug substance, suitable pharmaceutical formulations of tipifarnib for administration in tablets contain the following inactive ingredients: lactose monohydrate, maize starch, hypromellose, microcrystalline cellulose, crospovidone, colloidal anhydrous silica, and magnesium stearate. These are safe and well tested excipients that are commonly used in marketed products. The film coatings of the tablets contain hypromellose, titanium dioxide, lactose monohydrate, polyethylene glycol, and triacetin. “Tipifarnib derivative” as used herein refers not only to R208176 but also to molecules closely structurally related to either tipifarnib or R208176 with similar pharmacologic activity against HDV; this term includes, for example and without limitation, compounds described in U.S. Pat. Nos. 6,169,096; 6,365,600; 6,420,387; 6,734,194; 6,743,805; 6,838,467; and 7,253,183.

Tipifarnib derivative R208176, also known as “JNJ-17305457”, refers to the FTase inhibitor (R)-1-(4-chlorophenyl)-1-[5-3-chlorophenyl)tetrazolo[1,5-a]quinazolin-7-yl]-1-(1-methyl-1H-imidazol-5-yl)methaneamine (and pharmaceutically acceptable salts and solvates thereof) having the structure shown below:

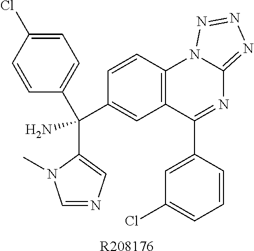

R208176 is an off-white, non-hygroscopic and crystalline powder. It has a molecular weight of 501.4 Daltons and a molecular formula of C₂₅H₁₈Cl₂N₈. R208176 is practically insoluble in water, citrate-NaOH buffer pH 6, borate-HCl buffer pH 8, borate-KCl—NaOH buffer pH 10, 0.1 N NaOH, intestinal fluid; very slightly soluble in citrate-HCl buffer pH 4; slightly soluble in citrate-HCl buffer pH 2; sparingly soluble in 0.1 N HCl and gastric fluid. Practically insoluble: <0.1 mg/mL; very slightly soluble: 0.1 to 1 mg/mL; slight soluble: 1-10 mg/mL; sparingly soluble: 10-33 mg/mL.

The term “HDV-RNA viral load” of a human serum or plasma sample refers to the number of copies of human HDV-RNA in a given amount of human serum or plasma sample. Currently, in the United States, there is one commercially available test for the qualitative detection of HDV-RNA and HDV antibody (Quest Therapeutics), but no commercially available clinical tests for the quantitation of HDV-RNA in clinical samples. However, several such assays reported in the literature (e.g., Kodani et al. 2013 J. Virol. Methods, 193(2), 531; and Karatayli et al, 2014, J. Clin. Virol, 60(1), 11) utilize a quantitative real-time reverse transcription-polymerase chain reaction (qRT-PCR) assay for quantification of HDV-RNA in serum or plasma suitable for use in accordance with the methods of the invention. The amount of signal generated during the assay is proportional to the amount of HDV-RNA in the sample. The signal from the test sample is compared to that of a dilution series of a quantified Hepatitis Delta RNA standard, and a copy number of genome copies is calculated.

The term “HDV infection” with respect to a human (host) refers to the fact that the host is suffering from HDV infection. Typically, an HDV infected human host will have a viral load of HDV-RNA of at least about 2 log HDV-RNA copies/mL of host serum or plasma or 10² copies of HDV-RNA/mL of host serum or plasma, often at least about 3 log HDV-RNA copies/mL of host serum or plasma or 10³ copies of HDV-RNA/mL of host serum or plasma, and, often, especially for patients not on any therapy, at least about 4 log HDV-RNA copies/mL of host serum or plasma or 10⁴ copies of HDV-RNA/mL of host serum or plasma, such as about 4 log HDV-RNA copies/mL of host serum or plasma to 7 log HDV-RNA copies/mL of host serum or plasma or 10⁴-10⁷ copies of HDV-RNA/mL of host serum or plasma.

The terms “host,” “subject,” “patient,” or “organism” include humans and mammals (e.g., mice, rats, pigs, cats, dogs, and horses). Typical hosts to which compounds of the present disclosure may be administered will be mammals, particularly primates, especially humans. For veterinary applications, a wide variety of subjects will be suitable, e.g., livestock such as cattle, sheep, goats, cows, swine, and the like; poultry such as chickens, ducks, geese, turkeys, and the like; and domesticated animals particularly pets such as dogs and cats. For research applications, a wide variety of mammals will be suitable subjects, including rodents (e.g., mice, rats, hamsters), rabbits, primates, and swine such as inbred pigs and the like. The term “living host” refers to a host noted above or another organism that is alive and refers to the entire host or organism and not just a part excised (e.g., a liver or other organ) from the living host.

The term “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, intravenous, buccal, rectal, parenteral, intraperitoneal, intradermal, intracheal, intramuscular, subcutaneous, inhalational and the like.

The terms “pharmaceutically acceptable excipient,” “pharmaceutically acceptable diluent,” “pharmaceutically acceptable carrier,” or “pharmaceutically acceptable adjuvant” means an excipient, diluent, carrier, and/or adjuvant that are useful in preparing a pharmaceutical composition that are generally safe, non-toxic and neither biologically nor otherwise undesirable, and include an excipient, diluent, carrier, and adjuvant that are acceptable for veterinary use and/or human pharmaceutical use. “A pharmaceutically acceptable excipient, diluent, carrier and/or adjuvant” as used in the specification and claims includes one and more such excipients, diluents, carriers, and adjuvants.

The term “pharmaceutically acceptable salt” refers to those salts that retain the biological effectiveness and optionally other properties of the free bases and that are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like. In the event that embodiments of the disclosed agents form salts, these salts are within the scope of the present disclosure. Reference to an agent of any of the formulas herein is understood to include reference to salts thereof, unless otherwise indicated. The term “salt(s)”, as employed herein, denotes acidic and/or basic salts formed with inorganic and/or organic acids and bases. In addition, when an agent contains both a basic moiety and an acidic moiety, zwitterions (“inner salts”) may be formed and are included within the term “salt(s)” as used herein. Pharmaceutically acceptable (e.g., non-toxic, physiologically acceptable) salts are preferred, although other salts are also useful, e.g., in isolation or purification steps which may be employed during preparation. Salts of the compounds of an agent may be formed, for example, by reacting the agent with an amount of acid or base, such as an equivalent amount, in a medium such as one in which the salt precipitates or in an aqueous medium followed by lyophilization. Embodiments of the agents that contain a basic moiety may form salts with a variety of organic and inorganic acids. Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides (formed with hydrochloric acid), hydrobromides (formed with hydrogen bromide), hydroiodides, 2-hydroxyethanesulfonates, lactates, maleates (formed with maleic acid), methanesulfonates (formed with methanesulfonic acid), 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, toluenesulfonates such as tosylates, undecanoates, and the like. Embodiments of the agents that contain an acidic moiety may form salts with a variety of organic and inorganic bases. Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines (formed with N,N-bis(dehydroabietyl)ethylenediamine), N-methyl-D-glucamines, N-methyl-D-glucamides, t-butyl amines, and salts with amino acids such as arginine, lysine, and the like. Basic nitrogen-containing groups may be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and others. Solvates of the agents of the disclosure are also contemplated herein. To the extent that the disclosed active compounds, and salts thereof, may exist in their tautomeric form, all such tautomeric forms are contemplated herein as part of the present disclosure. To the extent that the disclosed active compounds, and salts thereof, may exist as their N-oxides, all such N-oxides are contemplated herein as part of the present disclosure; methods of preparing such N-oxides are within the skill of one in the art.

The term “therapeutically effective amount” as used herein refers to that amount of an embodiment of the agent (which may be referred to as a compound, an inhibitory agent, and/or a drug) being administered that will treat to some extent a disease, disorder, or condition, e.g., relieve one or more of the symptoms of the disease, i.e., infection, being treated, and/or that amount that will prevent, to some extent, one or more of the symptoms of the disease, i.e., infection, that the host being treated has or is at risk of developing.

The terms “treatment”, “treating”, and “treat” are defined as acting upon a disease, disorder, or condition with an agent to reduce or ameliorate the pharmacologic and/or physiologic effects of the disease, disorder, or condition and/or its symptoms. “Treatment,” as used herein, covers any treatment of a disease in a host (e.g., a mammal, typically a human or non-human animal of veterinary interest), and includes: (a) reducing the risk of occurrence of the disease in a subject determined to be predisposed to the disease but not yet diagnosed as infected with the disease, (b) impeding the development of the disease, and/or (c) relieving the disease, i.e., causing regression of the disease and/or relieving one or more disease symptoms. “Treatment” is also meant to encompass delivery of an inhibiting agent to provide a pharmacologic effect, even in the absence of a disease or condition. For example, “treatment” encompasses delivery of a disease or pathogen inhibiting agent that provides for enhanced or desirable effects in the subject (e.g., reduction of pathogen viral load, reduction of disease symptoms, etc.).

The term “unit dosage (or dose) form,” as used herein, refers to physically discrete units suitable as unitary dosages for human and/or animal subjects, each unit containing a predetermined quantity of a compound (e.g., an anti-viral compound, as described herein) calculated in an amount sufficient to produce the desired treatment effect in association with a pharmaceutically acceptable diluent, carrier or vehicle. In other words, “unit dosage form,” as used herein, refers to physically discrete units suitable as unitary dosages for human and/or animal subjects, each unit containing a pharmaceutically acceptable composition of a predetermined quantity of a compound. The specifications for unit dosage forms depend on the particular compound employed, the route and frequency of administration, and the effect to be achieved, and the pharmacodynamics associated with each compound in the host.

All deuterated analogs (a compound is a deuterated analog of another compound, the “parent compound”, if it differs from the parent compound by only replacement of one or more hydrogen atoms with one or more deuterium atoms) of any active pharmaceutical ingredient described herein, including without limitation, tipifarnib, R208176, ritonavir, and cobicistat, are, for purposes of the present invention, encompassed by reference to the parent compound.

All stereoisomers of any agent described herein, including without limitation, tipifarnib, R208176, ritonavir, cobicistat, and any other active pharmaceutical agent described herein, such as those that may exist due to asymmetric carbons on the various substituents, including enantiomeric forms (which may exist even in the absence of asymmetric carbons) and diastereomeric forms, are contemplated within the scope of this disclosure. Individual stereoisomers of the compounds of the disclosure may, for example, be substantially free of other isomers, or may be admixed, for example, as racemates or with all other, or other selected, stereoisomers. The stereogenic centers of the compounds of the present disclosure can have the S or R configuration as defined by the IUPAC 1974 Recommendations.

### II. HDV Treatment

The present invention provides methods for treating diseases relating to HDV infection. HDV always presents as a co-infection with HBV, but a co-infected patient is much more likely to die of complications of viral infection than a patient infected with HBV alone. Currently available anti-HBV agents include the following HBV reverse transcriptase inhibitors, including but not limited to nucleotide or nucleoside reverse transcriptase (RT) inhibitors: Lamivudine, Adefovir, Entecavir, Telbivudine, Clevudine, and Tenofovir. HBV/HDV co-infection may be treated with alpha interferon therapy or therapy with pegylated interferon alpha 2a (alone or in combination with one of the foregoing HBV reverse transcriptase inhibitors). In accordance with the methods of this invention, tipifarnib or R208176 or another tipifarnib derivative is administered alone or in combination with another prenyltransferase inhibitor or other therapeutic for the treatment of HBV and/or HDV infection, including treatment in combination with one or more of the foregoing HBV reverse transcriptase inhibitors and/or interferon and/or myrcludex and/or ritonavir or cobicistat (see the combination therapy section, below). In one embodiment, the subject is not known to have cancer and/or is not known to be infected with any virus other than HDV and HBV for which treatment is required.

In an embodiment, tipifarnib or R208176 or another tipifarnib derivative is used in combination with an effective amount of another agent such as an interferon to treat HDV infection. In an embodiment, an effective amount of tipifarnib or R208176 or another tipifarnib derivative is an amount that, when administered in one or more doses to a human in need thereof, reduces HDV viral load in the individual by at least about 1 log HDV-RNA copies/mL of host serum or plasma (or 10¹-fold), about 1.5 log HDV-RNA copies/mL of host serum or plasma (or 10^{1.5}-fold), about 2 log HDV-RNA copies/mL of host serum or plasma (or 10²-fold), about 2.5 log HDV-RNA copies/mL of host serum or plasma (10^{2.5}-fold), or about 3 log HDV-RNA copies/mL of host serum or plasma (or 10³-fold) or more, compared to the viral load in the individual not treated with tipifarnib or R208176 or another tipifarnib derivative.

HDV can severely damage the liver of infected patients. Accordingly, the present invention provides methods for preventing liver damage and, in some patients, restoring liver function. Thus, in some embodiments, an effective amount of tipifarnib or R208176 or another tipifarnib derivative is an amount that, when administered in one or more doses to a host (e.g., human) in need thereof, increases liver function in the individual by at least about 10%, at least about 25%, at least about 50%, at least about 75%, at least about 90%, or more, compared to the liver function in the individual not treated with tipifarnib or R208176 or another tipifarnib derivative. In other embodiments, the effective amount of tipifarnib or R208176 or another tipifarnib derivative and or an agent administered in combination with it is an amount that, when administered in one or more doses to a host (e.g., a human) in need thereof, reduces liver fibrosis in the host by at least about 10%, at least about 25%, at least about 50%, at least about 75%, at least about 90%, or more, compared to the degree of liver fibrosis in the individual not treated with tipifarnib or R208176 or another tipifarnib derivative.

Liver fibrosis reduction is determined by analyzing a liver biopsy sample. An analysis of a liver biopsy comprises assessments of two major components: necroinflammation assessed by “grade” as a measure of the severity and ongoing disease activity, and the lesions of fibrosis and parenchymal or vascular remodeling as assessed by “stage” as being reflective of long-term disease progression. See, e.g., Brunt, 2000, Hepatol. 31:241-246; and METAVIR (1994) Hepatology 20:15-20. Based on analysis of the liver biopsy, a score is assigned. A number of standardized scoring systems exist which provide a quantitative assessment of the degree and severity of fibrosis. These include the transient elastography, METAVIR, Knodell, Scheuer, Ludwig, and Ishak scoring systems.

### III. Pharmaceutical Compositions and Unit Dose Forms

The present invention provides pharmaceutical compositions comprising, or consisting essentially of, or consisting of tipifarnib or R208176 or another tipifarnib derivative and optionally one or more other anti-viral agents as identified herein and formulated with one or more pharmaceutically acceptable excipients, diluents, carriers and/or adjuvants. In addition, embodiments of the pharmaceutical compositions of the present invention include tipifarnib or R208176 or another tipifarnib derivative formulated with one or more pharmaceutically acceptable auxiliary substances. In particular, tipifarnib or R208176 or another tipifarnib derivative can be formulated with one or more pharmaceutically acceptable excipients, diluents, carriers, and/or adjuvants to provide an embodiment of a pharmaceutical composition of the invention.

A wide variety of pharmaceutically acceptable excipients are known in the art. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical 7^{th} Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In pharmaceutical dosage forms, tipifarnib or R208176 or another tipifarnib derivative may be administered in the form of its pharmaceutically acceptable salts, or pharmaceutically acceptable solvates of tipifarnib or R208176 or another tipifarnib derivative and its salts, or may be used alone or in appropriate association, as well as in combination, with other pharmaceutically active compounds. The following pharmaceutical formulations, unit dose forms, methods for their preparation, and excipients are merely exemplary and are in no way limiting.

For oral preparations, tipifarnib or R208176 or another tipifarnib derivative can be used alone or in pharmaceutical formulations of the invention comprising, or consisting essentially of, or consisting of tipifarnib or R208176 or another tipifarnib derivative in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

In one embodiment, the pharmaceutical formulation of the invention contains tipifarnib or R208176 or another tipifarnib derivative formulated for oral administration. In various embodiments, the unit dose form useful in the methods of the invention contains 100 mg, 150 mg, 200 mg, 250 mg and 300 mg of free base equivalent of tipifarnib. In various embodiments, the unit dose form useful in the methods of the invention contains 20 mg of free base equivalent of R208176. If a salt or a solvate is used, equivalently larger amounts will be required as is readily understood by the skilled artisan.

Pharmaceutical formulations and unit dose forms suitable for oral administration are particularly useful in the treatment of chronic conditions and therapies in which the patient self-administers the drug. For acute infections and life-threatening conditions, particularly those requiring hospitalization, intravenous formulations are desirable, and the present invention provides such formulations as well.

The invention provides pharmaceutical formulations in which tipifarnib or R208176 or another tipifarnib derivative can be formulated into preparations for injection in accordance with the invention by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Unit dosage forms for oral administration, such as syrups, elixirs, and suspensions, may be provided wherein each dosage unit, for example, teaspoonful, tablespoonful, tablet or suppository, contains a predetermined amount of the composition containing tipifarnib or R208176 or another tipifarnib derivative. Similarly, unit dosage forms for injection or intravenous administration may comprise in a composition as a solution in sterile water, normal saline or another pharmaceutically acceptable carrier. Appropriate amounts of the active pharmaceutical ingredient for unit dose forms of tipifarnib or R208176 are provided above.

Thus, the invention provides a variety of pharmaceutical formulations, unit dose forms, and drug delivery devices for administering tipifarnib or R208176 or another tipifarnib derivative in accordance with the methods of the invention. These include, but are not limited to, tablets, capsules, suspensions, and slow-release formulations suitable for oral administration.

### IV. Administration

As is clear from the previous section, the present invention provides methods and compositions for the administration of tipifarnib or R208176 or another tipifarnib derivative alone or in combination with an interferon, to a human for the treatment of HDV infection. In various embodiments, these methods of the invention span almost any available method and route suitable for drug delivery, including in vivo and ex vivo methods, as well as systemic and localized routes of administration. Generally, however, tipifarnib or R208176 or another tipifarnib derivative is administered orally. Typical oral administration schedules for these schedules are QD or BID administration schedules. For patients in which GI side effects are expected or have been demonstrated to be problematic, however, or for convenience, the methods of the invention can be practiced using patch technology, particularly patch technology that employ micro-needles, to administer the drug subcutaneously, and thereby avoid or at least ameliorate GI and other side effects.

In various embodiments of the methods of the invention, tipifarnib or R208176 or another tipifarnib derivative will be administered orally on a continuous, daily basis, at least once per day (QD), and in various embodiments two (BID) or three (TID) times a day. Typically, the therapeutically effective daily dose will be 100-300 mg tipifarnib or 20 mg R208176, administered BID.

Thus, in one embodiment, tipifarnib is administered orally at a dose of 100 mg BID. In another embodiment, tipifarnib is administered orally at a dose of 150 mg BID. In another embodiment, tipifarnib is administered orally at a dose of 200 mg BID. In another embodiment, tipifarnib is administered orally at a dose of 250 mg BID. In another embodiment, tipifarnib is administered orally at a dose of 300 mg BID. In one embodiment, R208176 is administered orally at a dose of 20 mg BID. Treatment is continued on a continuous daily basis for at least two to three months. In some embodiments, treatment is continued for at least six months to one year. In other embodiments, treatment is continued for the rest of the patient's life or until administration is no longer effective in maintaining the virus at a sufficiently low level to provide meaningful therapeutic benefit.

Dosing of tipifarnib or R208176 or another tipifarnib derivative can be accomplished in accordance with the methods of the invention using capsules, tablets, oral suspension for oral administration. Various combination therapies of the invention for the treatment of HDV infection are described in Section V, below.

A proof of concept (POC) trial demonstrating the antiviral effect of tipifarnib or R208176 or another tipifarnib derivative against HDV can be conducted in a cohort of 15 to 25 patients with chronic HDV infection. Patients will undergo pre-study screening, which may include the following assessments: liver biopsy within one-year of study enrollment; hematological assessment and monitoring throughout the study; blood chemistry assessment and monitoring throughout the study; screening for concomitant viral infections, including HBV, HCV, and HIV, as well as HDV viral loads; cancer assessment and screening, including liver carcinoma; patients co-infected with HCV, HIV, or who have received an experimental drug within the prior six months, or who have been recently diagnosed/treated for cancer may be excluded from the study to facilitate demonstration of improved health upon treatment as described herein.

Once a patient is qualified to enter the clinical trial, baseline HDV viral load levels will be determined. Patients will then receive active dosing with tipifarnib or R208176. For tipifarnib, a first cohort of patients may receive tipifarnib at a dose of 100 mg, 150 mg, 200 mg, 250 mg or 300 mg BID for at least 30 days. For R208176, a first cohort of patients may receive tipifarnib at a dose of 20 mg BID for at least 30 days. Dosing can be extended based on the treatment outcome, including e.g, the HDV viral load reduction.

HDV viral load levels can be assessed throughout the active therapy phase of the study, with heightened viral surveillance occurring at six time-points during the first 72 hours of therapy to gauge initial virologic response. Follow-up HDV viral load assessment will occur approximately every fourth day during the last 24 days of active therapy. Safety and pharmacokinetic data will be collected during the dosing phase, as well as examination of PBMC farnesyl transferase activity. In addition, patients will undergo post-treatment monitoring for six-months to assess HDV viral load as well as safety assessments.

### V. Combination Therapies

The pharmaceutically acceptable compositions or pharmaceutical formulations and unit dose forms described herein can be used in combination with interferons. Current medical practice to treat HBV infection and/or HBV and HDV co-infection sometimes employs either interferon-alpha monotherapy (including treatment with interferon-alpha-2b or a pegylated interferon, such as Pegasys, marketed by Roche, or PEG-Intron, marketed by Merck) or combination therapy with interferon-alpha and a nucleoside or nucleotide analogue, such as adefovir (Hepsera®), entecavir (Baraclude®), lamivudine (Epivir-HBV®, Heptovir®, Heptodin®), telbivudine (Tyzeka®), tenofivir (Viread®), and ribavirin (such as Rebetol® or Copegus®). In accordance with the methods of the present invention, tipifarnib or R208176 or another tipifarnib derivative is used in combination with one of these standard therapies to treat HDV infection (i.e., HBV and HDV co-infection).

### Interferons

Based on the type of receptor through which they signal, human interferons have been classified into three major types. In various embodiments, an interferon of any of Types I-III is used in combination with tipifarnib or R208176 or another tipifarnib derivative to treat HDV infection. All type I IFNs bind to a specific cell surface receptor complex known as the IFN-alpha receptor (IFNAR) that consists of IFNAR1 and IFNAR2 chains. The type I interferons present in humans are IFN-alpha, IFN-beta, IFN-epsilon, and IFN-omega. Type II IFNs bind to IFN-gamma receptor (IFNGR) that consists of IFNGR1 and IFNGR2 chains. The type II interferon in humans is IFN-gamma. The recently classified type III interferon group consists of three IFN-lambda molecules called IFN-lambda1, IFN-lambda2 and IFN-lambda3 (also called IL29, IL28A, and IL28B, respectively). These IFNs signal through a receptor complex consisting of IL10R2 (also called CRF2-4) and IFNLR1 (also called CRF2-12).

Thus, the present invention provides combination therapies in which an interferon-alpha or interferon-lambda are used in combination with tipifarnib or R208176 or another tipifarnib derivative. The term “interferon-alpha” or “IFN-α” and “interferon-lambda” or “IFN-λ” as used herein refers to a family of related polypeptides that inhibit viral replication and cellular proliferation and modulate immune response. The term “IFN-α” includes naturally occurring IFN-α; synthetic IFN-α; derivatized IFN-α (e.g., PEGylated IFN-α, glycosylated IFN-α, and the like); and analogs of naturally occurring or synthetic IFN-α. The term “IFN-α” also encompasses consensus IFN-α. Thus, essentially any IFN-α or IFNλ that has antiviral properties, as described for naturally occurring IFN-α, can be used in the combination therapies of the invention.

Suitable interferons for purposes of the invention include, but are not limited to pegylated IFN-α-2a, pegylated IFN-α-2b, consensus IFN and IFN-λ.

The term “IFN-α” encompasses derivatives of IFN-α that are derivatized (e.g., are chemically modified relative to the naturally occurring peptide) to alter certain properties such as serum half-life. As such, the term “IFN-α” includes IFN-α derivatized with polyethylene glycol (“PEGylated IFN-α”), and the like. PEGylated IFN-α, and methods for making same, is discussed in, e.g., U.S. Pat. Nos. 5,382,657; 5,951,974; and 5,981,709. PEGylated IFN-α encompasses conjugates of PEG and any of the above-described IFN-α molecules, including, but not limited to, PEG conjugated to interferon alpha-2a (Roferon, Hoffman La-Roche, Nutley, N.J.), interferon alpha-2b (Intron, Schering-Plough, Madison, N.J.), interferon alpha-2c (Berofor Alpha, Boehringer Ingelheim, Ingelheim, Germany); and consensus interferon as defined by determination of a consensus sequence of naturally occurring interferon alphas (Infergen®, InterMune, Inc., Brisbane, Calif.).

Thus, in some embodiments of the combination therapies of the invention, the IFN-α has been modified with one or more polyethylene glycol moieties, i.e., pegylated. Two forms of pegylated-interferon, peginterferon alfa-2a (40KD) (Pegasys, Hoffmann-La Roche) and peginterferon alfa-2b (12KD) (PegIntron, Merck), are commercially available, which differ in terms of their pharmacokinetic, viral kinetic, tolerability profiles, and hence, dosing.

Peginterferon alfa-2a (Pegasys) consists of interferon alfa-2a (˜20-kd) covalently linked to a 40-kd branched polyethylene glycol (PEG). The PEG moiety is linked at a single site to the interferon alfa moiety via a stable amide bond to lysine. Peginterferon alfa-2a has an approximate molecular weight of 60,000 daltons. The biologic activity of peginterferon-alfa-2a derives from its interferon alfa-2a moiety which impacts both adaptive and innate immune responses against certain viruses. This alpha interferon binds to and activates human type 1 interferon receptors on hepatocytes which activates multiple intracellular signal transduction pathways, culminating in the expression of interferon-stimulated genes that produce an array of antiviral effects, such as blocking viral protein synthesis and inducing viral RNA mutagenesis. Compared with the native interferon alfa-2a, the peginterferon alfa-2a has sustained absorption, delayed clear. Peginterferon alfa-2a is used as a fixed weekly dose. Peginterferon alfa-2a has a relatively constant absorption after injection and is distributed mostly in the blood and organs.

Peginterferon alfa-2b (PegIntron) consists of interferon alfa-2b covalently linked to a 12-kd linear polyethylene glycol (PEG). The average molecular weight of the molecule is approximately 31,300 daltons. Peginterferon alfa-2b is predominantly composed of monopegylated species (one PEG molecule is attached to one interferon molecule), with only a small amount of dipegylated species. Fourteen different PEG attachment sites on the interferon molecule have been identified. The biologic activity of peginterferon-alfa-2b derives from its interferon alfa-2b moiety, which impacts both adaptive and innate immune responses against certain viruses. This alpha interferon binds to and activates human type 1 interferon receptors on hepatocytes which activates multiple intracellular signal transduction pathways, culminating in the expression of interferon-stimulated genes that produce an array of antiviral effects, such as blocking viral protein synthesis and inducing viral RNA mutagenesis. Compared with the native interferon alfa-2b, the peginterferon alfa-2b has sustained absorption, delayed clearance, and a prolonged half life. Peginterferon alfa-2b is used as a weekly dose based on the weight of the patient. Peginterferon alfa-2b has a rapid absorption and a wider distribution in the body.

The PEG molecule of a PEGylated IFN-α polypeptide is conjugated to one or more amino acid side chains of the IFN-α polypeptide. In an embodiment, the PEGylated IFN-α contains a PEG moiety on only one amino acid. In another embodiment, the PEGylated IFN-α contains a PEG moiety on two or more amino acids, e.g., the IFN-α contains a PEG moiety attached to two, three, four, five, six, seven, eight, nine, ten, eleven, twelve, thirteen, or fourteen different amino acid residues. IFN-α may be coupled directly to PEG (i.e., without a linking group) through an amino group, a sulfhydryl group, a hydroxyl group, or a carboxyl group.

Pegylated interferon has been used in the management of HDV as a monotherapy, albeit with clearance of HDV in no more than a quarter of those treated. A combination therapy provided by the invention comprises administering tipifarnib or R208176 or another tipifarnib derivative as provided herein as a direct antiviral agent with an immune modulator such as interferon (optionally in combination with other antiviral medications). Illustrative interferons include those discussed above. In one embodiment of these combination therapies, pegylated interferon alfa-2a (Pegasys) is administered weekly in dosages of 180 microgram (mcg) or 135 mcg (used for patients that react negatively to the higher dose) subcutaneously (SQ). In another embodiment of these combination therapies, pegylated interferon alfa-2b (PegIntron) is administered weekly in dosages of 1.5 mcg/kg/wk SQ. In other embodiments of these methods, alfa-interferons are used as follows: consensus interferon (Infergen) administered at 9 mcg to 15 mcg SQ daily or thrice weekly; interferon-alfa 2a recombinant administered at 3 MIU to 9 MIU SQ administered thrice weekly; interferon-alfa 2b (Intron A) recombinant administered 3 MIU to 25 MIU SQ administered thrice weekly; and pegylated interferon lambda (IL-28) administered at 80 mcg to 240 mcg SQ weekly.

The term “IFN-α” also encompasses consensus IFN-α. Consensus IFN-α (also referred to as “CIFN” and “IFN-con” and “consensus interferon”) encompasses, but is not limited to, the amino acid sequences designated IFN-con₁, IFN-con₂ and IFN-con₃ which are disclosed in U.S. Pat. Nos. 4,695,623 and 4,897,471; and consensus interferon as defined by determination of a consensus sequence of naturally occurring interferon alphas (e.g., Infergen®, Three Rivers Pharmaceuticals, Warrendale, Pa.). IFN-con₁ is the consensus interferon agent in the Infergen® alfacon-1 product. The Infergen® consensus interferon product is referred to herein by its brand name (Infergen®) or by its generic name (interferon alfacon-1). DNA sequences encoding IFN-con may be synthesized as described in the aforementioned patents or other standard methods. In an embodiment, the at least one additional therapeutic agent is CIFN.

The term “IFN-λ” encompasses IFN-lambda1, IFN -lambda2, and IFN-lambda3. These proteins are also known as interleukin-29 (IL-29), IL-28A, and IL-28B, respectively. Collectively, these 3 cytokines comprise the type III subset of IFNs. They are distinct from both type I and type II IFNs for a number of reasons, including the fact that they signal through a heterodimeric receptor complex that is different from the receptors used by type I or type II IFNs. Although type I IFNs (IFN-alpha/beta) and type III IFNs (IFN-lambda) signal via distinct receptor complexes, they activate the same intracellular signaling pathway and many of the same biological activities, including antiviral activity, in a wide variety of target cells.

In various embodiments of the combination therapies of the invention, fusion polypeptides comprising an IFN-α and a heterologous polypeptide are used. Suitable IFN-α fusion polypeptides include, but are not limited to, Albuferon-alpha™ (a fusion product of human albumin and IFN-α; Human Genome Sciences; see, e.g., Osborn et al., 2002, J. Pharmacol. Exp. Therap. 303:540-548). Also suitable for use in the present methods are gene-shuffled forms of IFN-α. See, e.g., Masci et al., 2003, Curr. Oncol. Rep. 5:108-113. Other suitable interferons include Multiferon (Viragen), Medusa Interferon (Flamel Technology), Locteron (Octopus), and Omega Interferon (Intarcia/Boehringer Ingelheim).

Thus, in various embodiments, tipifarnib is dosed in combination with an interferon to treat HDV infection in accordance with the invention. In various embodiments, the interferon is pegylated IFN alfa 2a or pegylated IFN alfa 2b. Suitable doses of tipifarnib/pegylated IFN alfa 2a are 100 mg BID/180 mcg QW, 150 mg BID/180 mcg QW, 200 mg BID/180 mcg QW, 250 mg BID/180 mcg QW and 300 mg BID/180 mcg QW. Suitable doses of tipifarnib/pegylated IFN alfa 2b are 100 mg BID/1.5 mcg/kg patient weight QW, 150 mg BID/1.5 mcg/kg patient weight QW, 200 mg BID/1.5 mcg/kg patient weight QW, 250 mg BID/1.5 mcg/kg patient weight QW and 300 mg BID/1.5 mcg/kg patient weight QW. Administration will be continuous for about 30 days, more typically 30 or 60 days, and often as long 6 months, 9 months, and 12 months.

Thus, in various embodiments, R208176 is dosed in combination with an interferon to treat HDV infection in accordance with the invention. In various embodiments, the interferon is pegylated IFN alfa 2a or pegylated IFN alfa 2b. Suitable doses of tipifarnib/pegylated IFN alfa 2a are 20 mg BID/180 mcg QW. Suitable doses of R208176/pegylated IFN alfa 2b are 20 mg BID/1.5 mcg/kg patient weight QW. Administration will be continuous for about 30 days, more typically 30 or 60 days, and often as long 6 months, 9 months, and 12 months.

### Boosting Agents

Pharmacokinetic “boosting” is the pharmacological enhancement of orally dosed drugs through the co-dosing with pharmacological enhancers which render these drugs more effective. Ritonavir (marketed under the trade name Norvir® by AbbVie, Inc.) is a pharmacologic enhancer, inhibiting two key stages of metabolism. First, it inhibits first-pass metabolism during absorption. Enterocytes that line the intestine contain both CYP3A4, one of the key cytochrome P450 isoenzymes associated with drug metabolism, and P-glycoprotein, an efflux transporter that can effectively pump drugs out of the gut wall and back into the intestinal lumen. Ritonavir inhibits both of these proteins and, consequently, may increase a coadministered drug's Cmax. Second, ritonavir inhibits CYP3A4 in the liver, thereby maintaining a drug's plasma half-life. It is also possible that ritonavir inhibits P-glycoprotein found in CD4+ cells. As a result, less drug is transported back out of the cell, thereby increasing the drug's intracellular half-life.

Cobicistat (marketed under the tradename Tybost® by Gilead Sciences) is another potent inhibitor of CYP3A. As does ritonavir, it “boosts” blood levels of other substrates of this enzyme but, unlike ritonavir, it has no anti-HIV activity. In addition, while it has a pronounced effect on the enzyme system (CYP3A) responsible for breaking down certain drugs, it does not affect other enzyme systems used by many other medications which may contribute to numerous potentially harmful drug interactions. Cobicistat does not impair fat cell functions in vitro like Norvir® does, meaning that cobicistat may be less likely to count fat accumulation and insulin sensitivity problems as side effects. Cobicistat is useful in the combination therapies of the invention at its approved or any lower dose in combination with tipifarnib or R208176 or another tipifarnib derivative at any dose and dosing frequency described herein.

In one embodiment of these combination therapies with a CYP3A4 inhibitor, ritonavir (Novir®) is administered at 100 mg once daily, up to 50 mg twice daily, up to 300 mg twice daily and increased at 2 to 3 day intervals by 100 mg twice daily, up to 600 mg twice daily. In another embodiment of these combination therapies, cobicistat (Tybost®) is administered at 150 mg once daily. In these embodiments of the invention, tipifarnib or another tipifarnib derivative may be dosed at 100 mg QD, 100 mg BID, 150 mg QD, 150 mg BID, 200 mg QD, 200 mg BID, 250 mg QD, 250 mg BID, 300 mg QD, or 300 mg BID, optionally in combination with interferon as described above. Thus, suitable doses of tipifarnib/ritonavir include (all QD administration, all BID administration and combinations of QD and BID administration) 100 mg/50 mg, 100 mg/100 mg, 150 mg/50 mg, 150 mg/100 mg, 200 mg/50 mg, 200 mg/100 mg, 250 mg/50 mg, 250 mg/100 mg, 300 mg/50 mg, 300 mg/100 mg. In all of these embodiments, administration is continued at least for 30 days, more often at least 60 days, and typically at least 90 days, although longer duration of treatment, as described above, can be beneficial to some patients.

In one embodiment of these combination therapies with a CYP3A4 inhibitor, ritonavir (Novir®) is administered at 100 mg once daily, up to 50 mg twice daily, up to 300 mg twice daily and increased at 2 to 3 day intervals by 100 mg twice daily, up to 600 mg twice daily. In another embodiment of these combination therapies, cobicistat (Tybost®) is administered at 150 mg once daily. In these embodiments of the invention, R208176 may be dosed at 20 mg QD or 20 mg BID, optionally in combination with interferon as described above. Thus, suitable doses of R208176/ritonavir include (all QD administration, all BID administration and combinations of QD and BID administration) 20 mg/50 mg and 20 mg/100 mg. In all of these embodiments, administration is continued at least for 30 days, more often at least 60 days, and typically at least 90 days, although longer duration of treatment, as described above, can be beneficial to some patients.

### Other HDV Therapeutic Compounds

Myrcludex B is in development as an entry inhibitor inactivating the sodium-taurocholate cotransporting polypeptide (NTCP) receptor found in the basolateral membranes of hepatocytes. Myrcludex B, a synthetic N-acylated preSI-derived lipopeptide binds to the NTCP receptor, a sodium/bile acid cotransporter, thereby inhibiting HBV/HDV entry. Sodium/bile acid cotransporters are integral membrane glycoproteins that participate in the enterohepatic circulation of bile acids. Two homologous transporters are involved in the reabsorption of bile acids, one absorbing from the intestinal lumen, the bile duct, and the kidney with an apical localization (SLC10A2), and the other being found in the basolateral membranes of hepatocytes (SLC10A1; NTCP). In various embodiments, Myrcludex B is used in combination with tipifarnib to treat HDV infection.

### Other Therapeutic Compounds

Other therapeutic compounds that may be administered with beneficial effect to an HDV-infected patient that is being treated in accordance with the invention include a nucleoside or nucleotide analog; a thiazolide; a protease inhibitor; a polymerase inhibitor; a helicase inhibitor; a Class C CpG toll-like receptor 7 and/or 9 antagonist; an amphipathic helix disruptor or NS4B inhibitor; a statin or other HMG CoA reductase inhibitor; an immunomodulator; an anti-inflammatory; a second prenylation inhibitor; a cyclophilin inhibitor; and an alpha-glucosidase inhibitor.

### Compounds Used to Treat HBV

In various combination therapies of the invention, tipifarnib or R208176 or another tipifarnib derivative is combined with an antiviral medication directed against HBV. Anti-HBV medications that are currently approved, with the exception of interferons, inhibit reverse transcriptase and are nucleoside or nucleotide analogues. These medications, while effective against HBV, are not effective against HDV as they do not lower HBsAg, which HDV needs to replicate; however, when used in the combination therapies of the invention, improved patient outcomes can be achieved. Currently approved anti-HBV medications include: interferon alpha (Intron A®), pegylated interferon (Pegasys®), lamivudine (Epivir-HBV®, Zeffix®, or Heptodin®), adefovir dipivoxil (Hepsera®), entecavir (Baraclude®), telbivudine (Tyzeka®, Sebivo®), clevudine (Korea/Asia), tenofovir (Viread®). Truvada®, which is a combination of tenofovir and emtricitabine, is not yet approved but has been shown to be effective in reducing HBV viral titers in early clinical trials and is useful in the combination therapies of the invention.

The methods and compositions of the invention having now been described in detail, the following examples are provided to illustrate methods by which the anti-viral activity tipifarnib or R208176 or another tipifarnib derivative utilized in the invention can be demonstrated.

Activity against HDV can be demonstrated in vitro through cell-based assays assessing the cytotoxicity and EC₅₀ of tipifarnib alone, and then in combination with other antiviral compounds. The cell lines used for these assays may be laboratory-derived and/or patient-derived cell lines. The examples herein are put forth so as to provide those of ordinary skill in the art with an illustrative disclosure and description of how to perform the methods and use the compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere. Any methods and materials similar or equivalent to those described herein can also be used in the practice or testing of the present invention.

### VI. Gastrointestinal Modifying Therapies

The pharmaceutically acceptable compositions or pharmaceutical formulations and unit dose forms described herein can be used in combination with gastrointestinal modifying therapies. Current medical practice to treat gastrointestinal irritations sometimes employs anti-emetics, H2-receptor antagonists, proton pump inhibitors and anti-diarrheals. Anti-emetic therapies include 5-HT₃ antagonists (such as ondansetron (Zofran®), tropisetron (Navoban®), granisetron (Kytril®), palonosetron (Aloxi®), and dolasetron (Anzemet®)) and NK1 receptor antagonists (such as aprepritant (Emend®), casopitant, and fosaprepitant (Emend® IV)). H2-receptor antagonists include ranitidine (Zantac®), famotidine (Pepcid®), cimetidine (Tagamet®) and nizatidine (Axid®). Proton pump inhibitors include omeprazole (Prilosec®), omeprazole/sodium bicarbonate (Zegerid®), esomeprazole magnesium (Nexium®), esomeprazole strontium, lansoprazole (Prevacid®), dexlansoprazole (Dexilant®) and pantoprazole sodium (Protonix®). Anti-diarrheals include atropine/diphenoxylate (Lomotil®, Lonox®), loperamide HCl (Imodium®), and bismuth subsalicylate (Kaopectate®, Pepto-Bismol®). In accordance with the methods of the present invention, tipifarnib or R208176 is used in combination with at least one, but in most cases more than one, of these standard GI-modifying therapies to treat HDV infection and potential GI irritation. Amelioration of GI irritation (nausea, vomiting, diarrhea, etc) through adherence to a prophylactic GI cocktail of an anti-emetic, antacid (H2-receptor antagonist or proton pump inhibitor) and/or an anti-diarrheal will allow for continued compliance of patients while on tipifarnib or R208176 or another tipifarnib derivative therapy.

### 5-HT₃ Antagonists

The 5-HT₃ antagonists are a class of drugs that act as receptor antagonists at the 5-HT₃ receptor, a subtype of serotonin receptor found in several critical sites involved in emesis, including vagal afferents, the solitary tract nucleus (STN), and the area postrema itself. Serotonin is released by the enterochromaffin cells of the small intestine in response to chemotherapeutic agents and may stimulate vagal afferents (via 5-HT₃ receptors) to initiate the vomiting reflex. The 5-HT₃ receptor antagonists suppress vomiting and nausea by inhibiting serotonin binding to the 5-HT₃ receptors. The highest concentration of 5-HT₃ receptors in the central nervous system (CNS) are found in the STN and chemoreceptor trigger zone (CTZ), and 5-HT₃ antagonists may also suppress vomiting and nausea by acting at these sites.

In one embodiment of these GI modifying therapies, this therapy is a 5-HT₃ receptor antagonist. In one embodiment of these GI modifying therapies, ondansetron (Zofran®) is administered 30 minutes to two hours before the start of tipifarnib or R208176 or another tipifarnib derivative therapy at 8 mg once daily, up to 8 mg two times daily, up to 8 mg three times daily. In this embodiment, administration is continued at least for the duration of tipifarnib or R208176 treatment. In another embodiment of these GI modifying therapies, tropisetron (Navoban®) is administered as a six day course of one 5 mg ampule, given intravenously on day 1 immediately before administering of tipifarnib or R208176, followed by oral administration on days 2 to 6. In another embodiment of these GI modifying therapies, granisetron (oral Kytril®) is administered at 2 mg given up to one hour before the start of tipifarnib or R208176 therapy or 1 mg twice daily. In this embodiment, administration is continued at least for duration of tipifarnib or R208176 treatment. In another embodiment of these GI modifying therapies, palonosetron (Aloxi®) is administered 30 minutes before the start of tipifarnib or R208176 therapy as a single 0.25 mg intravenous dose, up to 0.75 mg IV once daily. In another embodiment of these GI modifying therapies, dolasetron (Anzemet®) is administered 30 minutes before start of tipifarnib or R208176 therapy as a single 12.5 mg intravenous dose, up to 1.8 mg/kg dose or a single 100 mg dose.

### NK-1 Receptor Antagonists

NK1 is a G protein-coupled receptor located in the central and peripheral nervous system. This receptor has a dominant ligand known as Substance P (SP). SP is a neuropeptide, composed of 11 amino acids, which sends and receives impulses and messages from the brain. It is found in high concentrations in the vomiting center of the brain, and results in a vomiting reflux when activated. NK-1 receptor antagonists block signals given off by NK1 receptors.

In one embodiment of these GI modifying therapies, this GI modifying therapy is an NK-1 receptor antagonist. In one embodiment of these GI modifying therapies, aprepritant (Emend®) is administered in combination with an 5-HT3 receptor antagonist and a corticosteroid as a three day treatment consisting of a 125 mg dose on day one given one hour before start of tipifarnib or R208176 or another tipifarnib derivative therapy, followed by an 80 mg dose on days two and three. In another embodiment of these GI modifying therapies, fosaprepitant (Emend® IV) is administered in combination with an 5-HT3 receptor antagonist and a corticosteroid (dexamethasone) as a single day treatment consisting of one 150 mg dose of fosaprepitant given up to 30 minutes before start of tipifarnib or R208176 therapy followed by a single 12 mg dose of dexamethasone and a single dose of a 5-HT3 receptor antagonist such as odansetron, up to a single 150 mg dose of fosaprepitant given up to 30 minutes before start of tipifarnib or R208176 therapy followed by a single 8 mg dose of dexamethasone and a single dose of a 5-HT3 receptor antagonist such as ondansetron on day one, and a single 8 mg dose of dexamethasone on days 2 through 4.

### H2-Receptor Antagonists

H2-Receptor antagonists are a class of drugs used to block the action of histamine on parietal cells (specifically the histamine H2 receptors) in the stomach, decreasing the production of acid by these cells. H2 antagonists are used in the treatment of dyspepsia.

In one embodiment of these GI modifying therapies, this GI modifying therapy is an H2-receptor antagonist. In one embodiment of these GI modifying therapies, ranitidine (Zantac®) is administered at a dose of 150 mg twice daily, up to 150 mg four times daily for the duration of tipifarnib or R208176 or another tipifarnib derivative therapy. In another embodiment of these GI modifying therapies, famotidine (Pepcid®) is administered at a dose of 40 mg once daily, up to 20 mg twice daily, up to 40 mg twice daily for the duration of tipifarnib or R208176 therapy. In another embodiment of these GI modifying therapies, cimetidine (Tagamet®) is administered at dose of 400 mg once daily, up to 800 mg once daily, up to 1600 mg once daily, up to 800 mg twice daily, up to 300 mg four times daily, up to 400 mg four times daily, up to 600 mg four times daily for the duration of tipifarnib or R208176 therapy. In another embodiment of these GI modifying therapies, nizatidine (Axid) is administered at dose of 150 mg once daily, up to 300 mg once daily, up to 150 mg twice daily for the duration of tipifarnib or R208176 therapy.

### Proton Pump Inhibitors

Proton pump inhibitors are a class of antisecretory compounds that suppress gastric acid secretion by specific inhibition of the H⁺/K⁺ ATPase enzyme system at the secretory surface of the gastric parietal cell. Because this enzyme system is regarded as the acid (proton) pump within the gastric mucosa, inhibitors of this system have been characterized as a gastric acid-pump inhibitors in that they block the final step of acid production. This effect is dose-related and leads to inhibition of both basal and stimulated acid secretion irrespective of the stimulus.

In one embodiment of these GI modifying therapies, this GI modifying therapy is a proton pump inhibitor (PPI). In one embodiment of these GI modifying therapies, omeprazole (Prilosec®) is administered in combination with an antiacid up to four days before the start of tipifarnib or R208176 or another tipifarnib derivative therapy at a dose of 20 mg once daily, up to 40 mg once daily for the duration of tipifarnib or R208176 therapy. In another embodiment of these GI modifying therapies, omeprazole/sodium bicarbonate (Zegerid®) is administered one or more days before the start of tipifarnib or R208176 therapy at a dose of 20 mg once daily, up to 40 mg once daily for the duration of tipifarnib therapy. In another embodiment of these GI modifying therapies, esomeprazole magnesium (Nexium®) is administered at least one hour before tipifarnib or R208176 treatment at dose of 20 mg once daily, up to 40 mg once daily, up to 40 mg twice daily for the duration of tipifarnib or R208176 therapy. In another embodiment of these GI modifying therapies, esomeprazole strontium is administered at least one hour before tipifarnib or R208176 treatment at dose of 24.65 mg once daily, up to 49.3 mg once daily, up to 49.3 mg twice daily, for the duration of tipifarnib or R208176 therapy. In another embodiment of these GI modifying therapies, lansoprazole (Prevacid®) is administered up to two hours before tipifarnib or R208176 therapy at a dose of 15 mg once daily of lansoprazole, up to 30 mg once daily, up to 60 mg once daily, up to 30 mg two times daily for a duration up to 14 days, up to 30 mg three times daily for the duration of tipifarnib or R208176 therapy. In another embodiment of these GI modifying therapies, dexlansoprazole (Dexilant®) is administered up to two hours before tipifarnib or R208176 therapy at a dose of 30 mg once daily of dexlansoprazole, up to 60 mg once daily for the duration of tipifarnib or R208176 therapy. In another embodiment of these GI modifying therapies, pantoprazole sodium (Protonix®) is administered up to seven days before tipifarnib or R208176 therapy at a dose of 40 mg once daily, up to 40 mg twice daily for the duration of tipifarnib or R208176 therapy.

### Anti-Diarrheal Agents

There are two types of antidiarrheal drugs, those that thicken the stool and those that slow intestinal spasms. Thickening mixtures (such as psyllium) absorb water. This helps bulk up the stool and make it more firm. Antispasmodic antidiarrheal products slow the spasms of the intestine by acting on the μ-opioid receptors in the myenteric plexus of the large intestine. By decreasing the activity of the myenteric plexus, which in turn decreases the tone of the longitudinal and circular smooth muscles of the intestinal wall, the amount of time substances stay in the intestine increases, allowing for more water to be absorbed out of the fecal matter. Anti-spasmodics also decrease colonic mass movements and suppress the gastrocolic reflex.

In one embodiment of these GI modifying therapies, this therapy is an anti-diarrheal. In one embodiment of these GI modifying therapies, atropine/diphenoxylate (Lomotil®, Lonox®) is administered at a dose of two Lomotil tablets four times daily or 10 ml of Lomotil® liquid four times daily (20 mg per day) until initial control has been achieved, after which the dosage may be reduced to as little as 5 mg (two tablets or 10 ml of liquid) daily. In another embodiment of these GI modifying therapies, loperamide HCl (Imodium®) is administered at a dose of 4 mg (two capsules) followed by 2 mg (one capsule) after each unformed stool, up to16 mg (eight capsules). In another embodiment of these GI modifying therapies, bismuth subsalicylate (Kaopectate®, Pepto-Bismol®) is administered as 2 tablets or 30 mL every 30 minutes to one hour as needed, up to eight doses in 24 hours.

### EXAMPLES

### Example 1

### Treatment of HDV-Infected Patients with Tipifarnib (R115777)

A previous study of 13 cancer patients identified 300 mg tipifarnib administered orally BID as the MTD for continuous dosing for up to 56 days. Dose limiting toxicities were granulocytopenia and neuropathy with 4 of 13 patients stopping treatment due to adverse events (grade 3 or 4). No DLTs were observed for 200 mg BID dose when dosed for 200 days. Most common AEs reported to be gastrointestinal-related.

To demonstrate in vivo efficacy of tipifarnib or a tipifarnib derivative in treating patients chronically infected with HDV, as documented by presence of HDV-RNA in patient serum at least 6 months before dosing, 12 patients chronically-infected with HDV are treated with either 200 BID tipifarnib or 250 mg BID tipifarnib for 24 weeks. Efficacy is demonstrated by either 1) >1 log drop of HDV-RNA in patient serum after 4 weeks, becoming completely, or nearly, undetectable after 24 weeks of treatment, or 2) >1 log drop of HDV-RNA in patient serum after 4 weeks with concomitant normalization of ALT values by week 12.

### Example 2

### Treatment of HDV-Infected Patients with Tipifarnib (R115777) in Combination with Ritonavir

Results from studies such as that described in Example 1 may indicate that additional efficacy may be required for significant therapeutic benefit, at least in some patients. For such patients, significant therapeutic benefit may be achieved in accordance to the invention by administering tipifarnib or a tipifarnib derivative in combination with a boosting agent. The use of a boosting agent can enable patients to achieve significant therapeutic benefit by increasing the patient serum concentration of tipifarnib and increasing exposure of tipifarnib to the liver.

To demonstrate in vivo efficacy of tipifarnib in combination with ritonavir in treatment of patients infected with HDV, 12 patients chronically-infected with HDV are treated with any of the following: 200 mg BID tipifarnib with 100 mg QD ritonavir, 250 mg BID tipifarnib with 100 mg QD ritonavir, 100 mg QD tipifarnib with 100 mg QD ritonavir, 150 mg QD tipifarnib with 100 mg QD ritonavir, 200 mg QD tipifarnib with 100 mg QD ritonavir or 250 mg QD tipifarnib with 100 mg QD ritonavir for 24 weeks. Efficacy is demonstrated by either 1) >1 log drop of HDV-RNA in patient serum after 4 weeks, becoming completely, or nearly, undetectable after 24 weeks of treatment, or 2) >1 log drop of HDV-RNA in patient serum after 4 weeks with concomitant normalization of ALT values by week 12.

### Example 3

### Treatment of HDV-Infected Patients with a Fixed-Dose Combination of Tipifarnib (R115777) and Ritonavir

To demonstrate in vivo efficacy of a fixed-dose combination of tipifarnib and ritonavir in a treatment of patients infected with HDV, patients chronically-infected with HDV are treated with either a tablet or capsule containing 100-300 mg tipifarnib with 100 mg ritonavir taken once daily or a tablet or capsule containing 200-250 mg tipifarnib with 50 mg ritonavir taken twice daily for 24 weeks. Efficacy is demonstrated by either 1) >1 log drop of HDV-RNA in patient serum after 4 weeks, becoming completely, or nearly, undetectable after 24 weeks of treatment, or 2) >1 log drop of HDV-RNA in patient serum after 4 weeks with concomitant normalization of ALT values by week 12.

### Example 4

### Treatment of HDV-Infected Patients with Tipifarnib (R208176)

To demonstrate in vivo efficacy of R208176 in treating patients infected with HDV, clinical studies are conducted as in Example 1, but with a dose of 20 mg BID R208176 taken for 24 weeks.

### Example 5

### Treatment of HDV-Infected Patients with Tipifarnib (R208176) in Combination with Ritonavir

To demonstrate in vivo efficacy of R208176 in combination with ritonavir in patients infected with HDV, clinical studies are conducted as in Example 2, but with a dose of 20 mg QD or BID R208176 with 100 mg QD ritonavir taken for 24 weeks.

### Example 6

### Treatment of HDV-Infected Patients with a Fixed-Dose Combination of Tipifarnib (R208176) and Ritonavir

To demonstrate in vivo efficacy of a fixed-dose combination of R208176 and ritonavir in a treatment of patients infected with HDV, clinical studies are conducted as in Example 3, but with either a tablet or capsule containing 20 mg R208176 with 100 mg ritonavir taken once daily or a tablet or capsule containing 20 mg 208176 with 50 mg ritonavir taken twice daily for 24 weeks.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The invention has been described broadly and generically herein. Each of the narrower species and subgeneric groupings falling within the generic disclosure also form part of the invention. In addition, where features or aspects of the invention are described in terms of Markush groups, those skilled in the art will recognize that the invention is also thereby described in terms of any individual member or subgroup of members of the Markush group.

1. A method of treating a hepatitis delta virus (HDV) infection in a human, said method comprising administering to the human in need of such treatment a daily dose of about 100 mg BID, 150 mg BID, 200 mg BID, 250 mg BID or about 300 mg BID of tipifarnib for at least about 60 days, thereby treating the HDV infection.

2. A method of treating a hepatitis delta virus (HDV) infection in a human, said method comprising administering to the human in need of such treatment:
a daily dose of about 100 mg BID, 150 mg BID, 200 mg BID, 250 mg BID or about 300 mg BID of tipifarnib, and
a therapeutically effective amount of interferon-α,
for at least about 30 days, thereby treating the HDV infection.

3. A method of treating a hepatitis delta virus (HDV) infection in a human, said method comprising administering to the human in need of such treatment:
a daily dose of about 100 mg BID or QD, 150 mg BID or QD, 200 mg BID or QD, 250 mg BID or QD or about 300 mg BID or QD of tipifarnib, and
a therapeutically effective amount of a CYP3A4 inhibitor,
for at least about 30 days, thereby treating the HDV infection.

4. The method of claim 3, wherein the CYP3A4 inhibitor is selected from the group consisting of ritonavir and cobicistat.

5. A method of reducing hepatitis delta virus ribonucleic acid (HDV-RNA) in a human infected with HDV, said method comprising administering to the human a daily dose of about 100 mg BID, 150 mg BID, 200 mg BID, 250 mg BID or about 300 mg BID of tipifarnib for at least about 60 days, whereby the viral load of HDV is reduced by at least 1 log HDV-RNA copies/mL.

6. The method of reducing an hepatitis delta virus ribonucleic acid (HDV-RNA) in a human, said method comprising administering to the human in need of such reduction:
a daily dose of about 100 mg BID, 150 mg BID, 200 mg BID, 250 mg BID or about 300 mg BID of tipifarnib and
a therapeutically effective amount of interferon-α,
for at least about 30 days, whereby the viral load of HDV is reduced by at least 2 log HDV-RNA copies/mL.

7. The method of any one of claims 1-6, wherein tipifarnib is administered at a dose of 100 mg BID (200 mg per day).

8. The method of any one of claims 1-6, wherein tipifarnib is administered at a dose of 150 mg BID (300 mg per day).

9. The method of any one of claims 1-6, wherein tipifarnib is administered at a dose of 200 mg BID (400 mg per day).

10. The method of any one of claims 1-6, wherein tipifarnib is administered at a dose of 250 mg BID (500 mg per day).

11. The method of any one of claims 1-6, wherein tipifarnib is administered at a dose of 300 mg BID (600 mg per day).

12. The method of claim 3 or 4, wherein ritonavir is dosed at 100 mg QD.

13. The method of claim 12, wherein tipifarnib is dosed at 100 mg BID.

14. The method of claim 12, wherein tipifarnib is dosed at 100 mg QD.

15. The method of claim 12, wherein tipifarnib is dosed at 150 mg QD.

16. The method of claim 12, wherein tipifarnib is dosed at 150 mg BID.

17. The method of claim 12, wherein tipifarnib is dosed at 200 mg QD.

18. The method of claim 12, wherein tipifarnib is dosed at 200 mg BID.

19. The method of claim 12, wherein tipifarnib is dosed at 250 mg QD.

20. The method of claim 12, wherein tipifarnib is dosed at 250 mg BID.

21. The method of claim 12, wherein tipifarnib is dosed at 300 mg QD.

22. The method of claim 12, wherein tipifarnib is dosed at 300 mg BID.

23. The method of claim 3 or 4, wherein the ritonavir is dosed at 50 mg BID.

24. The method of claim 23, wherein tipifarnib is dosed at 100 mg BID.

25. The method of claim 23, wherein tipifarnib is dosed at 100 mg QD.

26. The method of claim 23, wherein tipifarnib is dosed at 150 mg BID.

27. The method of claim 23, wherein tipifarnib is dosed at 150 mg QD.

28. The method of claim 23, wherein tipifarnib is dosed at 200 mg BID.

29. The method of claim 23, wherein tipifarnib is dosed at 200 mg QD.

30. The method of claim 23, wherein tipifarnib is dosed at 250 mg BID.

31. The method of claim 23, wherein tipifarnib is dosed at 250 mg QD.

32. The method of claim 23, wherein tipifarnib is dosed at 300 mg BID.

33. The method of claim 23, wherein tipifarnib is dosed at 300 mg QD.

34. The method of any of claims 1-33, wherein dosing is continuous for a period of at least 30 days to at least one year.

35. A method of treating a hepatitis delta virus (HDV) infection in a human, said method comprising administering to the human in need of such treatment a daily dose of 20 mg BID of R208176 for at least about 60 days, thereby treating the HDV infection.

36. A method of treating a hepatitis delta virus (HDV) infection in a human, said method comprising administering to the human in need of such treatment:
a daily dose of about 20 mg BID of R208176, and
a therapeutically effective amount of interferon-α,
for at least about 30 days, thereby treating the HDV infection.

37. A method of treating a hepatitis delta virus (HDV) infection in a human, said method comprising administering to the human in need of such treatment:
a daily dose of about 20 mg BID of R208176, and
a therapeutically effective amount of a CYP3A4 inhibitor,
for at least about 30 days, thereby treating the HDV infection.

38. The method of claim 37, wherein the CYP3A4 inhibitor is selected from the group consisting of ritonavir and cobicistat.

39. A method of reducing hepatitis delta virus ribonucleic acid (HDV-RNA) in a human infected with HDV, said method comprising administering to the human a daily dose of about 20 mg BID of R208176 for at least about 60 days, whereby the viral load of HDV is reduced by at least 1 log HDV-RNA copies/mL.

40. The method of reducing an hepatitis delta virus ribonucleic acid (HDV-RNA) in a human, said method comprising administering to the human in need of such reduction:
a daily dose of about 20 mg BID of R208176, and
a therapeutically effective amount of interferon-α,
for at least about 30 days, whereby the viral load of HDV is reduced by at least 2 log HDV-RNA copies/mL.
